(19) United States
(12) Patent Application Publication
(10) Pub. No.: US 2018/0318582 A1
(43) Pub. Date: Nov. 8, 2018

(54) Systems and Methods for Stimulating a Patient to Prevent Oxygen Desaturation

(71) Applicant: Medipines Corporation, Anaheim Hills, CA (US)

(72) Inventors: Steve Lee, Anaheim Hills, CA (US); Dipen Makadia, Anaheim, CA (US); Amy Fisher, Garden Grove, CA (US); Youngjae Lee, Rowland Heights, CA (US); Timothy Marcum, Anaheim, CA (US)

(21) Appl. No.: 15/965,587

(22) Filed: Apr. 27, 2018

Related U.S. Application Data
(60) Provisional application No. 62/501,677, filed on May 4, 2017.

(51) Int. Cl. A61N 1/36; A61B 5/1455; A61B 5/083; A61B 5/08; A61B 5/053; A61B 5/00

(52) U.S. Cl. CPC A61N 1/3601; A61B 5/14551; A61B 5/0836; A61B 5/0833; A61B 5/0826; A61B 5/0531; A61B 5/4818; A61B 5/6831; A61B 5/6826; A61B 5/746; A61B 5/7405; A61B 5/4836; A61B 5/6823

(57) ABSTRACT

There is provided a system including a controller, a breathing sensor securable to a patient to determine breathing data of the patient, a pulse oximetry sensor securable to the patient to sense pulse oximetry data of the patient, and a stimulator securable to the patient to stimulate the patient. The controller configured to execute instructions to obtain the breathing data from the breathing sensor, obtain the pulse oximetry data from the pulse oximetry sensor, determine, based on the breathing data of the patient and the pulse oximetry data of the patient, whether the patient is to be stimulated, and deliver, using the stimulator, stimulations to the patient, in response to determining that the patient is to be stimulated.

### RELATED APPLICATION(S)

The present application claims the benefit of and priority to a pending Provisional Patent Application Ser. No. 62/501,677, filed May 4, 2017, and titled “Systems and Methods for Detecting and Reversing Disordered Breathing,” which is hereby incorporated fully by reference into the present application.

### BACKGROUND OF THE INVENTION

Obstructive sleep apnea (OSA) is a sleep disorder where breathing is interrupted by repetitive upper airway closure during sleep, resulting in oxygen desaturation or low blood oxygen level. This condition affects as many as twenty percent of the U.S. adult population (estimated at 250 million as of 2016), and of those individuals, up to ninety percent are believed to be undiagnosed. OSA can be a critical issue for surgical patients in the days following surgery due to lingering effects of anesthesia that may depress their auto-response to recover from episodes of sleep apnea. Obstructive sleep apnea is typically defined as an airway closure that lasts more than ten seconds and occurs five times or more per hour.

Today, hospital patients are monitored with pulse oximeters that trigger an alarm when blood oxygen falls below a dangerous level. In advanced care environments, patients who have been diagnosed as having OSA receive a higher level of attention by the medical staff. However, a large number of patients who have not been diagnosed as having OSA are left especially vulnerable to low oxygen levels associated with post-surgical recovery.

Relying solely on the medical staff to intervene for frequent sleep apnea episodes of many patients in a hospital setting is unpractical. A system that relies on the medial staff to manually intervene for frequent sleep apnea episodes of many patients prevents the medical staff from performing other important and urgent tasks. With a projected shortage of nurses of up to 500,000 by 2025, the current approach to address sleep apnea will place a great strain on the limited hospital resources, and a better solution is needed.

### SUMMARY

There are provided systems and methods for stimulating a patient to prevent oxygen desaturation, substantially as shown in and/or described in connection with at least one of the figures, and as set forth more completely in the claims.

### BRIEF DESCRIPTION OF THE DRAWINGS

Various implementations are described below with reference to the accompanying drawings of which:

FIG. 1 is a schematic illustration depicting a breathing monitoring and arousal system operatively joined with a human patient.

FIG. 2 is a schematic illustration depicting a wrist unit.

FIG. 3 is a perspective view illustration depicting a patient-contacting side of an example wrist unit.

FIG. 4 is a perspective view illustration depicting internal components of the wrist unit of FIG. 3 with a top housing section removed.

FIG. 5 is a perspective view illustration depicting a top side of another example wrist unit.

FIG. 6 is a perspective view illustration depicting a patient-contacting side of the example wrist unit of FIG. 5.

FIG. 7 is a cut-away view illustration depicting internal components of the wrist unit of FIG. 5 with a top housing section removed.

FIG. 8 is a schematic block diagram illustrating components of an example breathing monitoring and arousal system.

FIG. 9 is a schematic block diagram illustrating components of an electrical stimulation sub-system of an example breathing monitoring and arousal system.

FIG. 10 is a schematic drawing depicting components of an example breathing monitoring and arousal system.

FIG. 11 is a schematic drawing depicting patient-interacting components of an example breathing monitoring and arousal system.

FIG. 12 is a schematic drawing depicting a respiration monitor belt for measuring variation of chest volume or abdomen volume during breathing.

FIG. 13 is a schematic graph illustrating various forms of patient data collected during breathing interruptions.

FIG. 14 is a schematic graph illustrating waveforms of breathing gases during steady-state breathing.

FIG. 15 is a schematic process flow chart illustrating an example process for determining whether to deliver arousal stimulation to a patient.

FIG. 16 is a schematic process flow chart illustrating an example process for determining whether to deliver arousal stimulation to a patient.

FIG. 17 is a schematic process flow chart illustrating an example process for determining whether to deliver arousal stimulation to a patient.

FIG. 18 is a schematic process flow chart illustrating an example process for determining whether to deliver arousal stimulation to a patient.

FIG. 19 is a schematic drawing depicting a wrist unit connected to peripheral components.

FIG. 20 illustrates a graphical user interface screen displaying a motion sensor calibration, which may be implemented for use with the breathing monitoring and arousal system.

FIG. 21 illustrates a graphical user interface screen displaying a live data capture from various sensors and stimulation events, which may be implemented for use with the breathing monitoring and arousal system.

### DETAILED DESCRIPTION

The various implementations will be described in detail with reference to the accompanying drawings. References made to particular examples and implementations are for illustrative purposes and are not intended to limit the scope of the invention or the claims. Various features of the different disclosed implementations can be combined to form further implementations, which are part of this disclosure.

The present disclosure describes various systems and methods for monitoring one or more parameters indicative of a patient's breathing and, if the parameters indicate that breathing has stopped or slowed unacceptably, producing a stimulation signal selected to arouse the patient.

As used herein, the term “patient” may refer to any human user of a monitoring system, whether or not such a person is under medical care while using the system. Additionally, in cases where a monitor is configured for veterinary use, the term “patient” may include non-human mammals using the system.

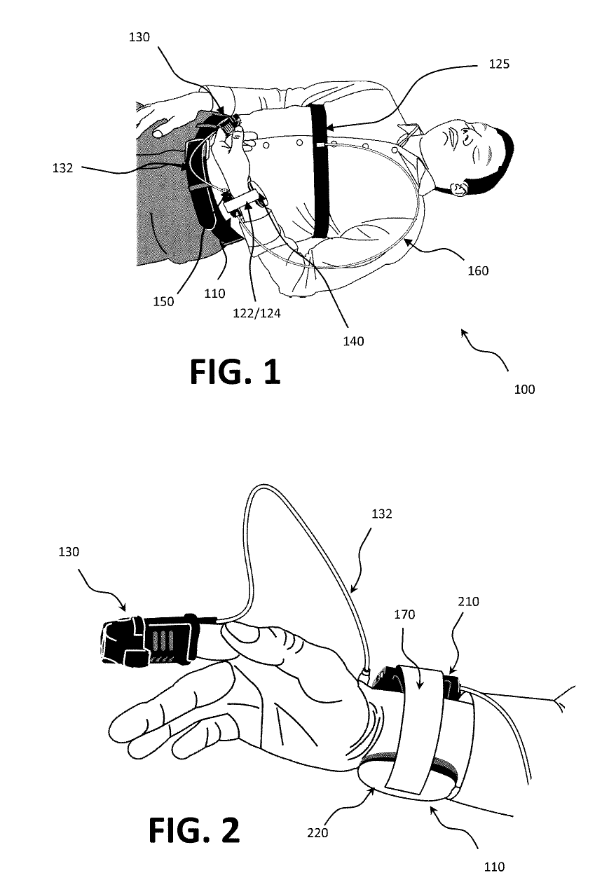

FIG. 1 provides a schematic illustration of an implementation of a monitoring and arousal system 100, which may comprise a wrist unit 110 containing a motion sensor securable to the patient, an SpO₂ circuit, a stimulator unit, a processor unit, a respiration monitor belt (e.g. a chest strap) attached or securable to a patient as a breathing sensor for providing breathing data, and/or a finger clip 130 attached or securable to a patient's finger. In some implementations, the finger clip may be replaced by other suitable pulse oximetry measurement devices, such as an ear clip configured to be secured to an ear lobe of the patient, a nose clip configured to be secured to a nose of the patient, etc.

In some implementations, the wrist unit 110, finger clip 130, and respiration monitor belt 125 may communicate with electronic devices such as a PC, tablet PC, and/or a smartphone via wires or cables. Alternatively, some or all of the components may communicate with other electronic devices wirelessly, such as via Bluetooth, WiFi, Zigbee, infrared, or any other suitable digital, radio, optical, or other wireless communications protocol.

As shown in FIG. 1, the wrist unit 110 may comprise a first cable connector 140 for receiving an electrical stimulator cable 122 and/or a signal cable 124 which may contain conductors for transmitting motion sensor signals. The wrist unit 110 may also include a finger cable connector 150 for a cable connecting the wrist unit 110 via a cable 132 to a finger clip 130 which may contain optical sources and sensors for performing pulse oximetry measurements when positioned on a patient's fingertip to provide pulse oximetry data. The respiration monitoring belt 125 may be connected to the wrist unit 110 via wires 160 for measuring lung activity or respiration rate. In some implementations, conductors for motion sensor signals, pulse oximetry signals, and stimulation signals may be carried in a single cable. In other implementations, a separate cable may be provided to carry electrical stimulation signals to be delivered to a patient's skin via electrodes incorporated into the wrist unit.

FIG. 2 illustrates a close-up view of a wrist unit 110, which has a processor unit 210 and stimulation unit 220, secured to a patient's wrist by a strap 170 passing through strap retaining structures such as one or more slots in the wrist unit housing.

In various implementations, one or more components of a controller (e.g., as described below) may be housed within the wrist unit 110. Components in the wrist unit 110 may communicate with other devices via wired or wireless communication. In various implementations, the wrist unit may take any suitable form, and may cover a larger or smaller portion of a patient's hand, arm, and/or wrist.

In some implementations, the finger clip 130 may communicate wirelessly directly with the wrist unit 110, in which case the cable 132 connecting the finger clip 130 to the wrist unit 110 may be omitted.

In some implementations, the wrist unit 110 may contain electronics for controlling pulse oximetry measurements taken with the finger clip 130 to provide pulse oximetry data. In such implementations, the wrist unit 110 may also contain electronics for communicating with other devices via cables or wirelessly using any suitable wireless communications protocol.

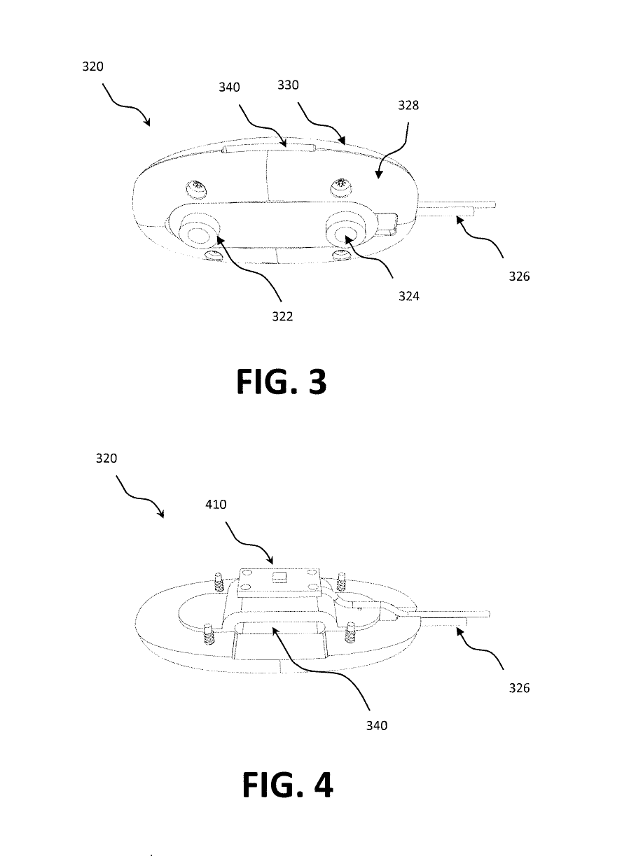

FIG. 3 illustrates an under-side of an implementation of a stimulation unit 320. The under-side of the wrist unit 320 may include a pair of stimulation electrodes 322, 324 and one side of a hook-and-loop closure, buckle, clip, clamp, adhesive tape, multiple straps, or other strap closure.

The stimulation unit 320 may be made in a clamshell structure with a top housing section 330 and a bottom housing section 328 secured together by mechanical fasteners such as latches, screws, bolts, adhesives, etc. Stimulation electrodes 322, 324 may extend outward from the bottom housing section 328, so as to positively contact a patient's skin when the wrist unit 320 is in place. One or more cables 326 may connect the stimulation unit 320 to a base unit (e.g., 110 in FIG. 1). In some implementations, the pair of stimulation electrodes on the wrist unit delivers electrical energy (“stimulus”) to cause “arousal” state by delivering safe low dosage in the form of milliamp current in certain frequency pulses.

An example of peripheral stimulation using oxygen saturation monitoring and using motion sensor is described in U.S. Pat. No. 8,417,351 (“the '351 patent”), which is incorporated herein by reference. In some implementations, peripheral nerve stimulation delivered to skin with increasing energy as appropriate to arouse obstructed breathing, as described in the '351 patent may be incorporated into monitoring to stimulate system.

As shown in FIG. 4 in which a top housing section 330 has been removed, the stimulation unit 320 may contain a motion sensor 410 housed within the stimulation unit 320. In some implementations, a strap retaining structure 340 may also extend through a central section of the stimulation unit 320.

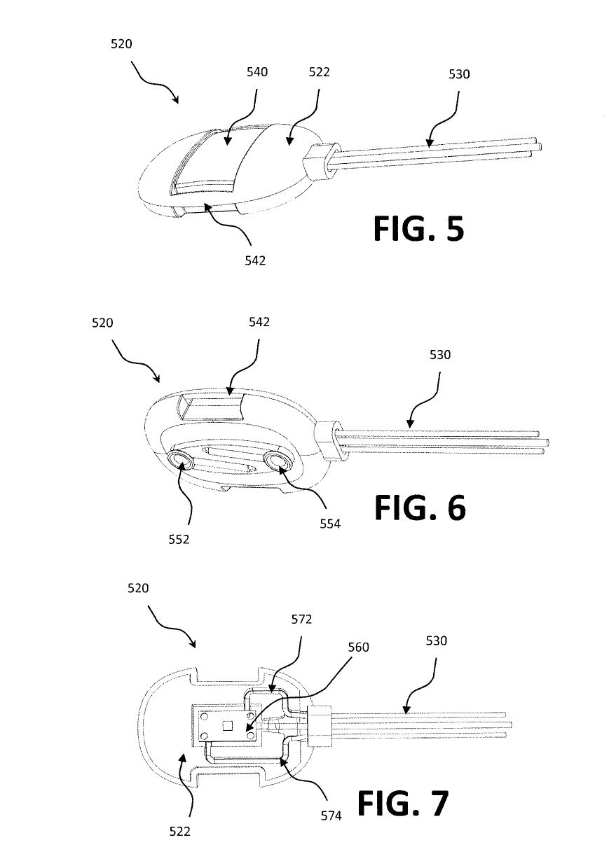

FIG. 5 through FIG. 7 illustrate another implementation of a wrist unit 520 including a housing 522, a cable or cable bundle 530, a strap retaining section 540 with strap retaining structures 542, stimulation electrodes 552, 554, and a motion sensor 560 within the housing 522. FIG. 7 also illustrates stimulation leads 572, 574 extending from the cable 530 to the stimulation electrodes 552, 554.

In various implementations, wrist units may be made of cleanable materials such as hard or soft plastics, metals, silicone, or other materials that may be sterilized between uses. In other implementations, wrist units may be made with non-sterilizable single-use materials and/or structures. Single-use materials may be safely disposed according to clinical practice.

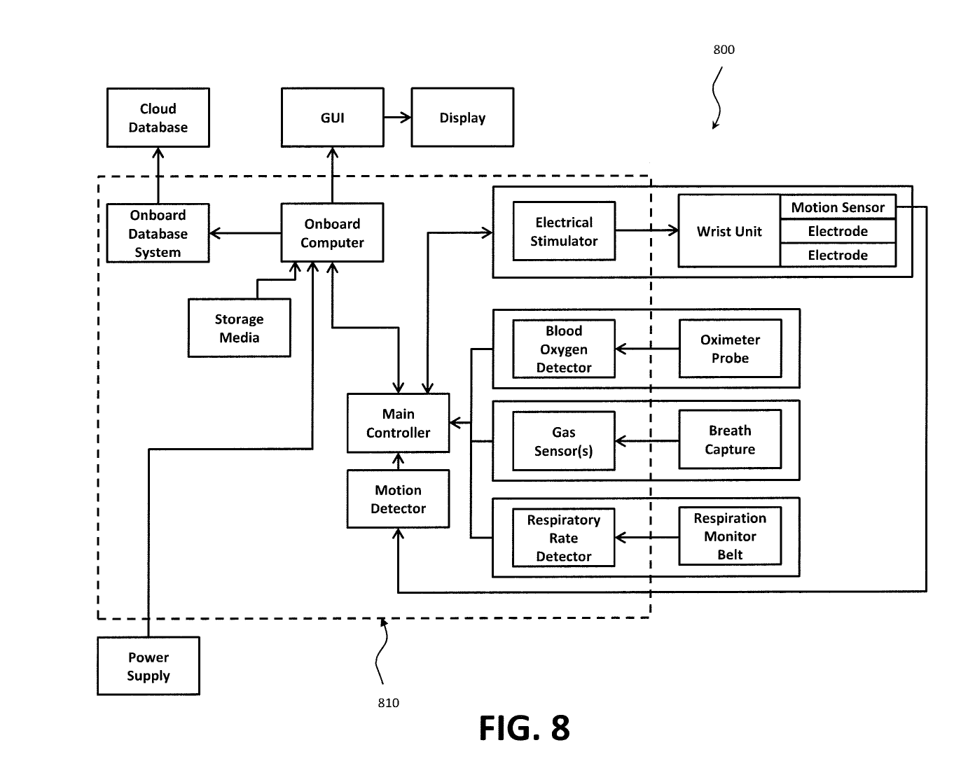

FIG. 8 illustrates components of a monitoring and arousal system 800 including components of a base unit controller 810 that may be included in a base unit, such as base unit 1010 in FIG. 10 or may be included in a wrist unit 110 or both, according to one implementation. In some implementations, the controller 810 may include a main controller, one or more storage media containing instructions to be executed by an onboard computer, a motion detector controller configured to evaluate signals received from the one or more motion sensors, a blood oxygen detector containing pulse oximetry electronics and instructions for evaluating signals received from an oximeter probe (e.g., finger clip), a respiratory rate detector acquired by using a respiration monitor belt (chest strap), a power supply, a user interface (e.g., GUI), and/or display device.

The system 800 may also include an electrical stimulator controller configured to deliver a pre-determined electrical stimulation signal to a patient via the electrodes in a wrist unit. The system 800 may also include an onboard database system for logging data collected during use of the system 800. In some implementations, an onboard database system may be configured to communicate via wired and/or wireless link with a “cloud database,” a hospital electronic medical records system (EMR), or other network-connected data storage device or service.

The power supply may comprise one or more batteries, a line voltage power supply, a transformer, voltage or current regulators, or any other suitable power supply components as needed. In some implementations, the user interface and/or display device may comprise a medical grade tablet, such as a CYBERMED T10 tablet, an AXIOMTEK tablet, or any other suitable display and/or user interface devices, such as a medical monitor or gas monitor.

In some implementations, the system 800 may also include gas exchange monitoring components which may include a breath capture device and one or more gas sensors. The gas sensors may include sensors for quantifying a partial pressure (or other measure) of component gases in air sampled from a patient's breathing. In various implementations, a pump may be used to draw a gas sample from a breath capture device into a gas sensor. Gas sensors may include oxygen sensors, carbon dioxide sensors, and/or others. Further examples of gas exchange monitoring systems are described herein below.

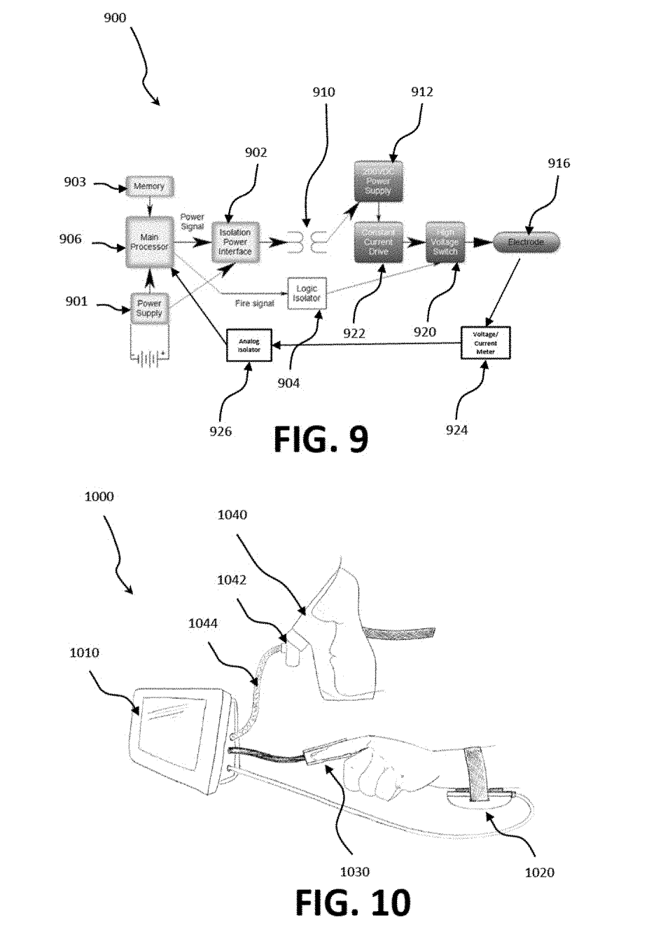

FIG. 9 illustrates components of an example electrical stimulator 900. The electrical stimulator 900 may include a power supply 901 and a memory device 903 which may be the power supply and storage media of the system 800. The electrical stimulator 900 may further include an isolation power interface 902 and a logic isolator 904 in communication with a main processor 906 which may include all or a portion of the onboard computer of the system 800, or the main processor 906 may be an independent processor. The isolation power interface 902 may be in communication with an isolation transformer 910, which may in turn be in communication with a high voltage power supply 912. The high voltage power supply 912 may be configured to deliver a high voltage electrical signal to the electrodes 916 of the stimulation unit via a high voltage switch 920 that may be controlled by a constant current drive 922 and the logic isolator 904. The main Processor 906 may obtain values of applied current and/or voltage from the electrodes 916 using a volt/current meter 924 through an analog isolator 926.

In various implementations, the main processor 906 may run the electrical stimulation algorithm 900 using oximetry data. Memory 903 is utilized to record data and for retrieving settings for stimulation. The main processor 906 may control the isolation power interface 902. When the patient needs to be stimulated, the main processor 906 turns power on to the isolation power interface 902, such that the power is delivered to the power supply 912. In one implementation, when stimulator power supply reaches 200 VDC, then the main processor 906 sends a signal via logic isolator 904, which controls the high voltage switch 920, which delivers a constant low current pulse to the patient. The duration is defined by the signal, which controls the high voltage switch 920. For example, the main processor 906 defines a repetition rate, e.g. 50 Hz, and duration of positive and negative stimulation pulses. The stimulator's core may be a constant current drive that can generate a required constant current in an exemplary range of 1 mA to 5 mA, which may be set by the main processor 906 via the logic isolator 904. For optimal patient compliance, a monophasic stimulation can be applied with a 25-75 Hz pulse train of 0.3-0.7 msec pulses with an amplitude of 2-3 mA range. Further, a maximum current level may be enforced to ensure patient safety.

In various implementations, the electrical stimulator 900 may be configured to deliver different electrical stimulation signals depending on the needs of a particular patient or clinical environment. For example, in one implementation, the electrical stimulator may be configured to deliver a variable voltage, constant current electrical pulse. In some implementations, the constant current may be user-selectable within a range of about 1 mA to about 10 mA and the voltage may be on the order of about 200 V. In some implementations, the electrical stimulator 900 may be configured to deliver a stimulation pulse of about 25-75 Hz with a pulse width of about 300-700 μs.

In some implementations, the electrical stimulator 900 may be configured to detect a resistance or impedance across the electrodes by delivering a small known test current and measuring an AC or DC voltage across the electrodes. Such a measurement may provide an indication of the resistance or impedance of the patient's skin and tissue through which an electrical stimulation signal is to be delivered.

If a detected impedance is outside of a desired range, then the electrical stimulator may be configured to not deliver stimulation even if the main processor instructs the electrical stimulator 900 to deliver a stimulus signal. For example, if the detected resistance or impedance (“R”) is less than a lower threshold (e.g., if R is less than about 1 kΩ), the patient's skin may be too wet or too conductive to safely deliver a stimulation signal, and the electrical stimulator may prevent delivery of a stimulus signal even when instructed to do so by another component of the system and may trigger an alarm signal and/or transmit a message indicating an error and/or suggest action to correct the problem.

Similarly, if the detected resistance or impedance is greater than an upper threshold (e.g., if R is greater than about 100 MΩ), the electrodes may be making inadequate contact with the patient's skin (e.g., the wrist unit may require adjustment). In such a case, the electrical stimulator may trigger an alarm signal and/or transmit a message indicating an error and/or suggest action to correct the problem.

In some implementations, the electrical stimulator 900 may be configured to deliver stimulation signals with monophasic waveforms, which have been found to be ideal in arousing patients suffering acute apneic episodes (e.g., caused by sleep apnea). In some implementations, the electrical stimulator 900 may be configured to deliver stimulation signals with biphasic or more complex waveforms instead of or in addition to mono phasic waveforms. In some implementations, a monitoring and arousal system may include user interface elements allowing a user to select a waveform, current, voltage, or other waveform characteristics. In some implementations, a monitoring and arousal system may include user interface elements allowing a user to select from a list of patient conditions, and the monitoring and arousal system may automatically select ideal stimulation characteristics based on the selection, stored data, and/or measurements obtained by the monitoring and arousal system.

In various implementations, a monitoring and arousal system may be configured to deliver other forms of stimulation instead of or in addition to electrical stimulation. Such other forms of stimulation may include audio signals (e.g., audible or inaudible sounds), visible signals (e.g., lights or visible messages), tactile signals (e.g., signals driving one or more vibrating elements, blowing air at a patient, or other mechanical actions), thermal signals (e.g., signals driving infrared heaters, hot/cold thermal blankets, or other thermal elements).

The measurement of oxygen (O₂) and/or carbon dioxide (CO₂) levels in the body can provide valuable indications of whether the body is properly receiving and processing oxygen and removing carbon dioxide from the blood. Such measurements can also be indicative of impaired respiratory function, disease, trauma, or other respiratory complications. Traditionally, gas levels in the body are measured by withdrawing arterial blood and testing the blood samples using an arterial blood gas test, typically referred to as an arterial blood gases (ABGs).

The need to draw and test blood samples substantially limits the frequency with which such tests may be performed. However, more frequent, or even continuous monitoring of respiratory status may dramatically benefit patients using a breathing monitoring and arousal system.

Some implementations of a monitoring and arousal system may incorporate a gas meter configured to directly measure one or more constituents of air inhaled and/or exhaled by the patient. An example of an alveolar gas meter configured to measure at least one alveolar gas level is described in U.S. Pat. No. 8,545,415 (“the '415 patent”), which is incorporated herein by reference. In some implementations, an alveolar gas meter such as that described in the '415 patent may be incorporated into a monitoring and arousal system. Additional examples of gas exchange monitors incorporated into monitoring and arousal systems are described in the following paragraphs.

FIG. 10 illustrates components of one example implementation of a monitoring and arousal system 1000 incorporating a breathing gas meter as part of a breathing sensor for providing breathing data. The system 1000 of FIG. 10 includes a base unit 1010 which may contain electronic control components such as those described above with reference to FIG. 8. In addition to a wrist unit 1020 and a finger clip 1030, the system 1000 may also include a breath capture device such as a face mask 1040 covering patient's nose and/or mouth.

The face mask breath capture device 1040 may incorporate a breathing tube 1042 configured to allow air exhaled by the patient to exit the mask space with little resistance, allowing for normal steady-state breathing while maintaining breathing gases within the mask space and within the breathing tube 1042. The breathing tube 1042 may be joined to the base unit 1010 by a sample collection conduit 1044.

In some implementations, the sample collection conduit 1044 may comprise a drying tube. A suitable drying tube may include as those commercially available from Perma Pure LLC of Toms River, N.J. A drying tube may be included to remove some or all water vapor present in ambient and/or exhaled air samples delivered to the gas sensors in order to improve the quality of partial pressure measurements reported by the gas analyzers.

In some implementations, a breath capture device may be configured for continuous or un-attended monitoring of a patient's breathing by joining a low-resistance breathing tube to a device securable to a patient in order to collect samples of exhaled air. For example, a breathing tube 1042 may be integrated with or replaced by a face mask 1040 as illustrated in FIG. 10. In some implementations, face masks 1040 adapted for use with a monitor system may include oxygen masks, face masks designed for use with CPAP (continuous positive airway pressure) or BiPAP (bi-level positive airway pressure) devices, or other related face mask devices.

Face masks 1040 may be adapted for use with a monitoring and arousal system by including a substantially resistance-free conduit 1042 joining an interior side of the mask to an exterior of the mask and through which the patient may inhale and exhale air while experiencing substantially minimal air-flow resistance relative to breathing without the mask. A sample collection tube (or “transport tube”) 1044 may join the substantially resistance-free conduit 1042 to an air-sample intake section of a base unit 1010. In some implementations, a sample collection conduit 1044 may be joined directly to the mask.

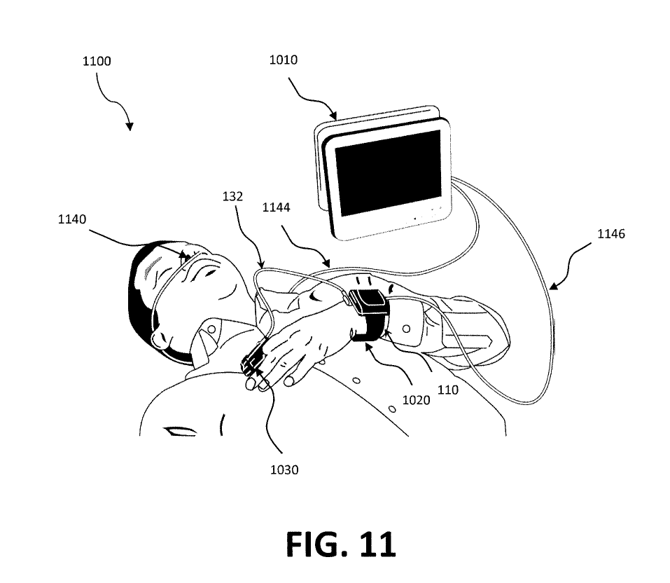

In some implementations, an example of which is illustrated in the system 1100 of FIG. 11, a breath capture device may include a nasal cannula 1140 coupled to a sample collection tube 1144 without a mask or breathing tube. In such implementations, samples of breathing air may be drawn directly from one or both of a patient's nostrils (which may effectively act as low-resistance breathing tubes).

In other implementations, a resistance-free conduit and sample collection tube may be configured to obtain samples of breathing air via a tracheostomy tube, an endotracheal tube, or other conduit through which a patient may breathe. In some implementations, a conduit may be considered “resistance free” if it applies no more than about one PSI.

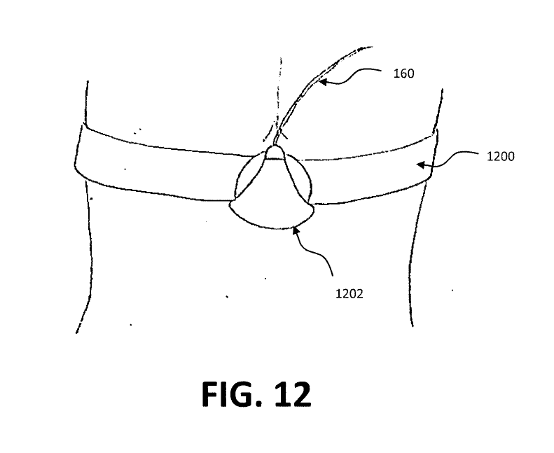

In other implementations, a respiration monitoring belt 1200 may be fastened around a patient's chest or abdomen that may measure chest or abdomen expansion during breathing. A possible implementation is displayed in FIG. 12. When a patient breathes, the chest expands, which may cause the belt to send signals to a monitor or computing system to indicate that the patient is breathing. The wrist unit may also read electrical or mechanical changes from the sensors in the belt to trigger stimulation. When a patient stops breathing, the belt does not send signals, indicating that the patient has stopped breathing and that there is a possible need for stimulation. The respiration monitoring belt may be used with any combination of a motion sensor, gas monitor, pulse oximetry sensor, and stimulation device for the purposes described herein.

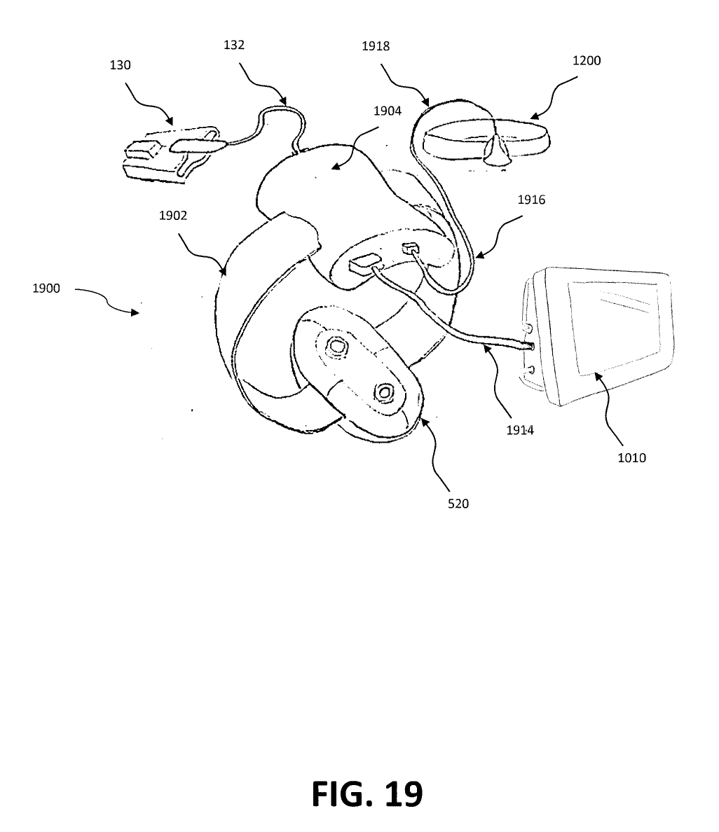

In some implementations, a wrist device 1900 may be worn by the patient, as shown in FIG. 19. The wrist unit 1900 may place a stimulation device 520 below the patient's wrist, a processor unit 1904 above the patient's wrist, and a connector band 1902 secures the wrist unit on the patient. The processor unit 1904 may house electronics, batteries, and algorithms that may respond to inputs and trigger metrics from other connections to decide whether to administer stimulation to the patient. The processor unit 1904 may receive inputs from a finger pulse oximeter 130, a wearable respiration monitor belt (chest strap) 1200, and a gas monitoring device 1010 to determine the current breathing state of the patient. The processor unit 1904 sends signals to the stimulating device when the patient stops breathing. The processor unit 1904 may use different algorithms depending on the combination of connectors.

A gas exchange measurement sub-system may include sample collection tube connected to carbon dioxide (CO₂) sensor and/or an oxygen (O₂) sensor. A pump may be included to draw gas samples from the sample collection tube, through the gas sensors, and eject tested air through exit conduits and out of the system through a vent. Signal carriers (e.g., wires, fiber optic cables, wireless communications devices etc.) may be provided to transmit signals from the gas sensors to an onboard computer or other data processing device.

Although a variety of different oxygen sensing devices could be employed as an O₂ sensor, in one implementation an oxygen sensor may include an Ultra-Fast Oxygen (UFO-130-2) sensor manufactured by Teledyne Analytical Instruments of the City of Industry, Calif. A CO₂ sensor may include a CO2WFA carbon dioxide sensor manufactured by Treymed Inc. or a Jaeger HCS CO₂ sensor manufactured by VIASYS Healthcare GmbH of Hoechberg, Germany or the Microstream® CO₂ sensor manufactured by Oridion Medical Inc. of Needham, Mass. Alternatively, any other suitable gas sensors may be used.

Additionally, other gas analyzers may also be included to measure quantities of additional gases in exhaled and/or inhaled air or ambient air. O₂ and CO₂ sensors may generally be configured to communicate digital or analog signals to a controller or other processor. In various implementations, the gas sensors may also include integrated electronics to perform some signal processing or other analysis prior to communicating a signal to a controller or other processor.

The gas exchange measurement sub-system may be configured to measure partial pressure of one or more gases in exhaled air. The partial pressure of a particular gas in a mixed-gas sample represents the hypothetical pressure of that particular gas if it alone occupied the same volume as the mixed-gas sample at the same temperature. Partial pressures are typically measured in units of millimeters of mercury (mm Hg), but may also be presented or used in any other units of pressure, such as atmospheres, bars, pounds per square inch (PSI), pascals (newtons per square meter), torr, etc.

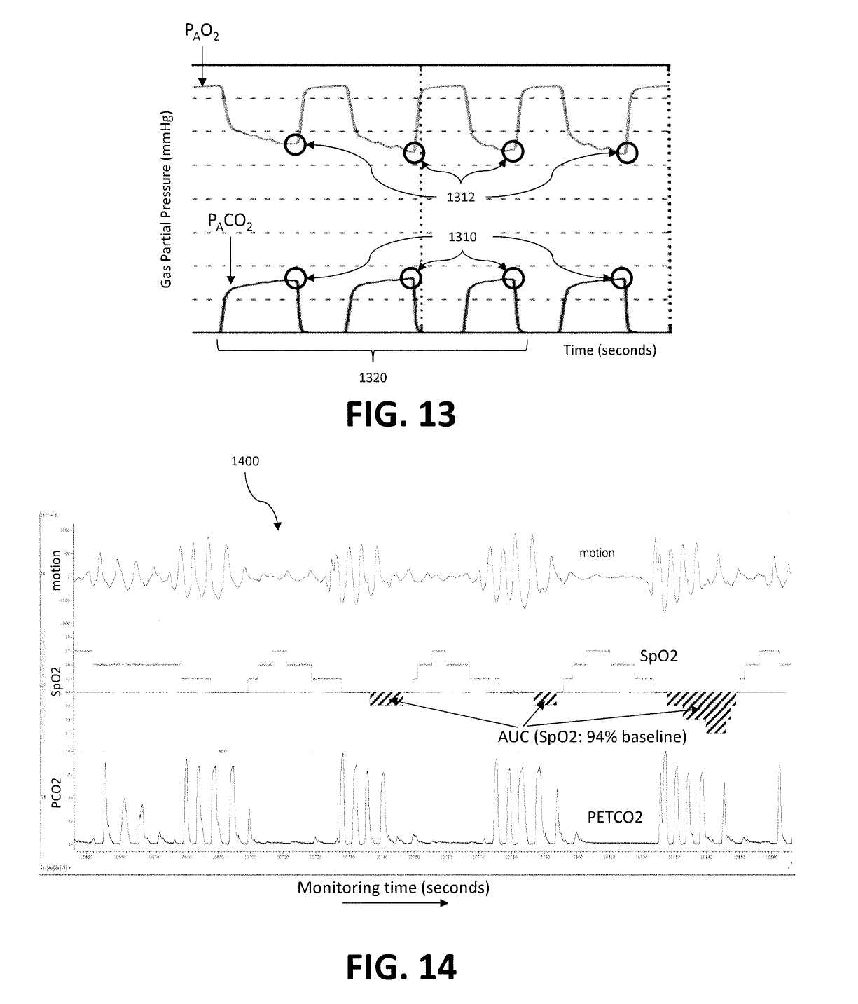

Some implementations may determine an “end-tidal value” for the partial pressure of alveolar carbon-dioxide (P_{A}CO₂) and/or an “end-tidal value” for the partial pressure of alveolar oxygen (P_{A}O₂) from sampled breathing gases obtained during each inhale/exhale breathing cycle. Determination of an end-tidal value may be understood with reference to FIG. 13, which illustrates a graph of example O₂ and CO₂ partial pressure data collected over a period of time. The lower curve in FIG. 13 represents a partial pressure of CO₂ throughout the breathing cycle. The upper curve represents a partial pressure oxygen throughout the breathing cycle. Both curves are shown during a number of inspiration/expiration breathing cycles. During each expiration cycle, PCO₂ (measured partial pressure of CO₂) rises up, comes to a plateau (which may have a rising slope), and then falls as the expiration cycle ends. Conversely, PO₂ (measured partial pressure of O2) falls, comes to a plateau (which may have a declining slope) and then rises as the expiration cycle ends. Partial pressures of CO₂ are lower than partial pressures of O₂ throughout normal breathing.

As used herein, the phrase “end-tidal value” refers to the value of a measured variable at the end of an expiration cycle (i.e., at the completion of exhalation of a tidal volume). Therefore, in some implementations, an end-tidal value of P_{A}CO₂ or P_{A}O₂ may be determined from a series of data points by identifying the end-of-cycle point in each cycle. For example, in FIG. 13 a peak 1310 at the end of each exhale cycle is indicated by a circle, corresponding to end-tidal P_{A}CO₂. In some implementations, the peak 1310 at the end of each exhale cycle may be identified by identifying a local maximum partial pressure value during each cycle.

Alternatively, the peak 1310 at the end of each exhale cycle may be identified by detecting a sudden change of slope, and identifying a peak immediately preceding the slope change. In some implementations, a combination of both peak-detection and slope-change-detection may be used to identify a local end-tidal value for each cycle. The same or similar techniques may also be used for determining a local end-tidal value of P_{A}O₂ for each expiration cycle, e.g., as illustrated by circles 1312 at the bottom of each O₂ cycle.

In some implementations, local end-tidal values may be obtained for a plurality of cycles, and the local results may be averaged to obtain normalized end-tidal value over a period of time. Alternatively, a normalized end-tidal value may be obtained based on a maximum of multiple cycles, a minimum of multiple cycles, a median of multiple cycles, a mean (average) of multiple cycles or other normalization method. In some implementations, a normalized end-tidal value may be obtained based on a pre-determined number of breathing cycles (e.g., 2, 3, 4, 5, 6 or more cycles). In other implementations, a normalized end-tidal value may be obtained based on an arbitrary number of cycles occurring within a predetermined time duration such as that illustrated by bracket 1320 in FIG. 13. In some implementations, a normalized end-tidal value may be the end-tidal value used in calculations, the end-tidal value used for reporting directly on a display screen, and/or the end-tidal value stored in a memory device. The same or similar techniques may be used for determining normalized values of P_{A}O₂ and P_{A}CO₂. In some implementations, the end tidal values may be used to calculate respiration rate to detect patient breathing.

The various controllers, computers, analyzers, and similar devices described herein may comprise any suitable computing machine which may include analog and/or digital signal processing components, such as field-programmable arrays (FPGAs), digital signal processors (DSPs), programmable logic controllers (PLCs), analog-to-digital converters, power management circuits or controllers, filters, amplifiers, timers, counters, or other devices as needed.

A computing machine may comprise data storage and processing components within which one or more sets or sequences of instructions may be executed to cause the machine to perform any one of the processes, methods, or calculations described herein, according to various example implementations. In some implementations, a computing machine may operate as a standalone device or may be connected (e.g., networked) to other machines. In a networked deployment, a computing machine may operate in the capacity of a server or a client machine in server-client network environments, or a machine may act as a peer machine in peer-to-peer (or distributed) network environments.

A computing machine may include a personal computer (PC), a laptop computer, a desktop computer, a server computer, a tablet PC, a hybrid tablet, a set-top box (STB), a personal digital assistant (PDA), a mobile telephone, a web appliance, a network router, switch or bridge, or any machine capable of executing instructions (sequential or otherwise) that specify actions to be taken by that machine. Further, even if only a single machine is illustrated, the term “machine” shall also be taken to include any collection of machines that individually or jointly execute a set (or multiple sets) of instructions to perform any one or more of the methodologies discussed herein. For example, in some cases, a single physical machine may be configured to operate as multiple virtual machines by separately allocating resources of a physical machine to multiple separate processes.

Various implementations of the system may obtain and use one or more of various trigger metrics to determine whether a stimulation signal should be delivered. The following paragraphs provide examples of some such trigger metrics. Example processes for using these or other metrics to determine whether to deliver a stimulus signal are described further below. In general, one or more actions may be taken if a trigger metric is determined to be outside of an acceptable range (e.g., above and/or below one or more acceptable threshold values). Actions taken when a trigger metric falls outside of an acceptable threshold may include the delivery of a stimulation signal, the start of a stimulation delivery process/algorithm (examples of which are described below), additional analysis, the start of an additional measurement, or other actions.

The pulse oximeter may generally measure oxygen saturation (typically abbreviated SpO₂), which is typically represented as a dimensionless percent. In some implementations, a measured SpO₂ value may be used directly based on established thresholds for acceptable oxygen saturation levels. Higher levels of oxygen saturation suggest more efficient breathing and oxygenation. Generally, a blood oxygen saturation of below about 90% is considered problematic, so in some implementations, an acceptable range of blood oxygen saturation threshold may be between about 89% and about 95%, and in some particular cases may be about 90%, 91%, 92%, 93%, or 94%. As described below, various actions may be taken if an oxygen saturation value below an acceptable threshold is detected.

In some implementations, two oxygen saturation thresholds may be used, where a first action (e.g., delivery of a stimulation signal or initiation of a stimulation delivery process) is taken if saturation below an upper threshold is detected, and a second action (e.g., sounding an audible alarm, initiation of an alarm process, or delivery of a different type of stimulus signal) may be taken if the saturation falls below a lower threshold. In some such implementations, an upper saturation threshold may be set above 90%, such as 95%, 94%, 93%, 92%, 91% (or fractional points within a similar range), and a lower threshold may be set at a value from about 90% down to about 85%.

In some implementations, an analysis of multiple measured oxygen saturation values may be used as a trigger metric. For example, in some embodiments, a result of an “area-under-the-curve” (AUC) analysis of SpO₂ measurements obtained over a period of time may be used as a trigger metric.

As used herein, an AUC analysis of a particular metric generally includes a summation of all measurements of that metric that fall outside of a pre-determined range within a pre-determined period of time. An AUC metric provides the benefit of accounting for both the magnitude of a measured metric and the duration of time during which the measured metric was outside of a desired range. Using such a metric in place of instantaneous measurements alone may provide a more robust representation of the measured metric. The severity of the apnea episode may be determined by the length of time that the body is deprived of a proper amount of oxygen. If there is a very low supply of oxygen (very low SpO₂), damage can occur faster, so capturing the effects of both SpO₂ levels and time in the AUC may provide a more comprehensive trigger metric to prevent damaging apnea episodes.

For example, FIG. 14 illustrates a graph 1400 showing an example of an AUC metric. Based on an SpO2 threshold of 94%, the measurements below the threshold are indicated as hatched sections. The total area between the threshold and the SpO₂ values measured below the threshold is the AUC metric value. The AUC metric value may be obtained in a number of ways depending on sampling rates and desired simplifications. For example, in the most complex case, mathematical curves may be fit to the data, an integral of the fit curves may be calculated, and the AUC may be calculated as a difference between the threshold and the integrated measurements. A simpler method may include assuming measured values are constant for one-second intervals (or taking averages of sub-second measurements to obtain one measurement per second), and simply summing the differences between the threshold and the measured values for each second during which a measured value is below the threshold.

In mathematical terms, a de-saturated area-under-the-curve metric (AUC_{desat}) may be obtained by: AUC_{desat}=Σ[(SpO2threshold−SpO2under-threshold)*t], where t is the amount of time being considered for the subtraction operation.

The total time duration for an AUC metric may be a fixed time period or a rolling (or trailing) time period. For example, an AUC desaturation metric may be calculated based on every second tracked over a monitoring period, such as a one-hour period or an eight-hour period. To use the example in FIG. 14, all the shaded areas below the threshold value over the entire timeframe would be added. In other examples, an AUC desaturation may only be tracked during the desaturation period below a defined threshold.

In some implementations, the AUC value may be reset to a zero value every time SpO2 rises above the designated low-end threshold value (e.g., 89% to 90%). In such implementations, AUC is not a running value over a time period but is calculated as an individual value each time SpO2 drops below the threshold value. In such implementations, such individual AUC periods may be summed or averaged over a longer period of time to obtain an aggregated value. For example, in some implementation, the system may also calculate and display an AUC Average (AUC_{AVG}) in the UI showing an average AUC value over an hour time period.

As discussed above, AUC_{desat} will be zero unless SpO₂ is below a threshold saturation value. Therefore, increasing values of AUC_{desat} above 0 indicates a worsening condition. As a result, in some implementations, any positive non-zero value of AUC_{desat} may be unacceptable. In other implementations, a maximum acceptable AUC_{desat} may be established at which action (e.g., stimulation or alarm condition) should be taken. The system may provide clinicians with an option to select the threshold for AUC_{desat} based on their own clinical experience. For example, if AUC values under 90% for OSA patients exhibit a range between 10 and 500%-seconds, an AUC_{desat} value greater than 10%-seconds may be an unacceptable value that may trigger an action or stimulation. In some implementations, a second range from 500 to 800%-seconds may be defined by clinicians as a more severe range that may trigger an additional stimulation or other action. In other implementations, AUC_{desat} values greater than 5%-seconds, 15%-seconds or 20%-seconds may be unacceptable. This value may be chosen based on additional clinical experience.

In some implementations, an oxygen saturation area (O₂A) metric may be calculated based on SpO2 values measured during a pre-determined monitoring time period such as one minute. O₂A may be calculated as the average of SpO2 values over a one minute time period:

O₂A=(ΣSpO₂)/n*60 seconds, where n is the number of SpO₂ samples per minute.

As with the AUC metric, O₂A may be updated at any pre-determined interval, such as every second, every 10 seconds, every 30 seconds, etc. In some implementations, an oxygen saturation area threshold may be established, and action may be taken if the O₂A value falls below the threshold or remains below the threshold for a pre-determined period of time.

In some implementations, a desaturation rate (DSR) may be calculated to represent a rate-of-change of an SpO2 measurement. Desaturation rate may be calculated as an instantaneous rate of change of SpO2 for a specified period of time. In one implementation, DSR may be calculated from the most recent SpO2 value and the SpO2 value obtained a pre-determined period of time earlier (e.g., two seconds in one example). DSR may be positive, indicating a rate of increasing saturation or negative indicating a rate of desaturation. In mathematical terms, DSR may be obtained by:

DSR=[SpO2ᵢ−SpO2₍ᵢ₋ₜ₎]/t

If, for example, SpO2 falls 2% over a 10 second period, the DSR value would be −0.2. Any value lower than a pre-established threshold DSR would be unacceptable and any value positive or greater than the threshold would be acceptable.

In some embodiments, an end-tidal partial pressure value of oxygen (PETO2) or carbon dioxide (PETCO2) may be used as a trigger metric. For example, an upper acceptable threshold value of PETCO2 may be established (e.g., about 40 mmHg to about 50 mmHg, or about 45 mmHg in one example), and action may be taken in response to detection of an PETCO2 value above the upper threshold. In other implementations, a lower acceptable threshold value of PETCO2 may be established (e.g., about 10 mmHg in one example), and action may be taken in response to detection of an end-tidal PETCO2 value below the lower threshold. Normal PETCO2 values may be between 30 and 45 mm Hg.

In some implementations, a low-end threshold value of measured PETCO2 may be established to provide an indication of whether or not the patient is breathing. While breathing, even inefficiently, a patient will exhale quantities of CO2 in excess of the atmospheric quantity of CO2. Therefore, if a quantity of CO2 above a low-end threshold is detected in air sampled from a breath collection device, then the patient is most likely breathing. Such a measurement may provide a more immediate binary indication of breathing than SpO2 which is subject to a physiologic delay of several seconds.

Similarly, a lower acceptable threshold value of PETO2 may be established (e.g., about 80 mm Hg for mild form of hypoxia, to 60 mmHg moderate, and 50 mm Hg for severe form), and action may be taken in response to detection of an end-tidal PETO2 value or an instantaneous measured PO2 value below the lower threshold. Such a lower threshold may be established to provide a binary indication of breathing and/or to provide a dynamic measure of breathing quality.

In some implementations, a trigger metric may include a simple count of a number of incidents during which a metric is measured outside of an acceptable range within a pre-determined time period. For example, a number of times an SpO₂ value falls below a threshold within a one-minute period may be counted, and if that number exceeds an acceptable threshold, action may be taken.

In various implementations, motion signals from one or more motion sensors may be used individually or in combination as trigger metrics. For example, signals received from each motion sensor may be continuously evaluated to determine whether the motion sensor signal meets criteria defining a “motion” event. In some implementations, each motion sensor may comprise a six-channel motion sensor indicating motion from accelerometers in x, y, and z dimensions as well as gyroscope indications of rotation about pitch, roll, and yaw axes. In some implementations, a motion event may occur if two or more of the channels exceed pre-determined thresholds.

In some implementations, some motion sensors may have different thresholds than other motion sensors based on the location of the motion sensors on the patient's body. For example, a motion sensor on a patient's sternum may be subject to significant movement that is not sufficient to meet a definition of a motion event for the purposes of the monitoring and arousal system. Therefore, thresholds may be notably higher for a sternum-mounted motion sensor than for a wrist-mounted motion sensor.

In various implementations, respiratory rate signals from the respiration monitor belt (chest strap) may be used individually or in combination with other signals as trigger metrics. For example, if respiration rate from the chest strap falls below a certain threshold, it may trigger a stimulation or other action through the processor unit or other external computing unit.

The various implementations of monitoring and arousal systems described herein may be used to monitor a patient's breathing and deliver stimulation to promote arousal in the event of a determination that the patient's breathing is disordered, impaired, or stopped based on one or more trigger metrics.

With a breath rate capture device, which can be a gas monitor and/or a respiration monitor belt, wrist unit, pulse oximetry sensor clip, and any additional motion sensors in place, the system may be operated to continuously collect and store measurement data for each of the sensors available. Measurement data may include breathing gas and/or breath rate data from carbon dioxide and/or oxygen sensors and/or respiration monitor belt, pulse oximetry data from the pulse oximetry sensor, and motion data from the one or more motion sensors. In some implementations, the collected and stored data may be used directly as trigger metrics or may be further analyzed to calculate one or more trigger metrics or parameters based on a portion of measurement data collected over a period of time, e.g., a trailing time period from a few seconds to a minute or more.

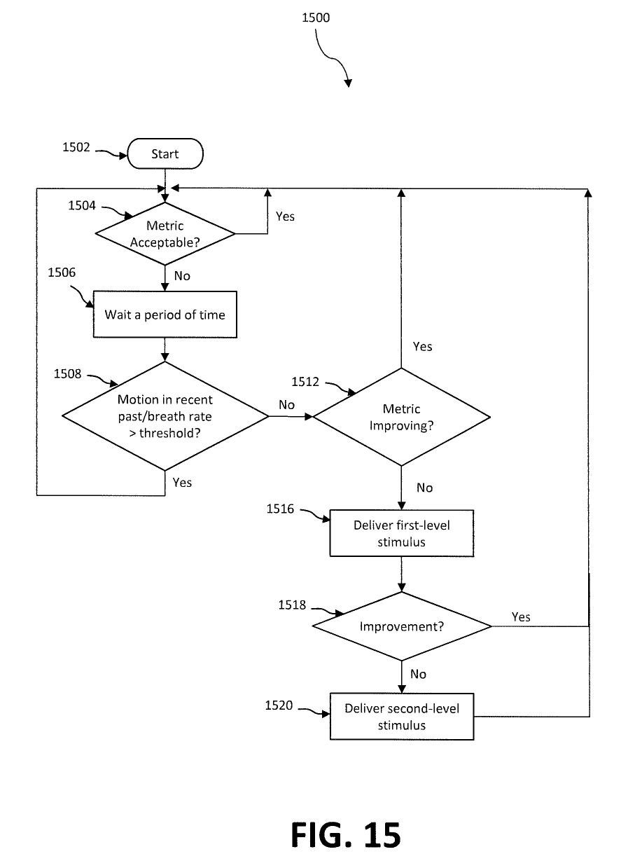

Once the trigger metrics or parameters have been collected and/or calculated, the system may perform a process to determine whether or not to deliver a stimulation signal to a patient. A process 1500 for monitoring a patient and delivering an arousal stimulation based on one trigger metric or parameter is illustrated in the process flow diagram of FIG. 15. The process 1500 of FIG. 15 may be executed as a software code by one or more processors and/or controllers within the base unit (e.g., 800 in FIG. 8), or any other suitably configured computer, processor, or controller.

The controller executing the process 1500 may begin at the start block 1502 and may proceed to evaluating a trigger metric. The trigger metric may be a direct measurement or a result of an analysis of multiple measured data points that is indicative of the quality of the patient's breathing, such as oxygen saturation (SpO2), an alveolar gas measurement, breath rate or a combination of these or other metrics. In some implementations, the trigger metric may be compared with a threshold value to determine whether the trigger metric is in a normal or acceptable range at block 1504. Examples of trigger metrics and acceptable ranges are described herein above.

If the trigger metric is determined to be within an acceptable range at 1504, then the controller may determine that the patient is breathing normally and may resume collecting data and monitoring the trigger metric. If the trigger metric is not within an acceptable range, then in some implementations, the controller may be instructed to wait a period of time at block 1506 in order to account for any physiological delay in the collection of data for the metric being used. During the waiting time period 1506, the controller may monitor a signal from the one or more motion sensors, gas sensors, and/or breath rate monitor. Once the period of waiting time has elapsed, the controller may, at block 1508, evaluate the motion sensor, gas, or breath rate signals obtained during the waiting time to determine whether motion was detected during the waiting time period (e.g., using, for example, the motion detector described above with reference to FIG. 8). In other implementations, the controller may continuously monitor signals from the one or more motion sensors, gas, and/or breath rate to determine whether motion has occurred without requiring a waiting period 1506.

In some implementations, the waiting time period 1506 may be a period of time between about one second and about 30 seconds or more. In some particular implementations, the waiting time period may be about 5, 10, 15, or 20 seconds.

If the controller determines at block 1508 that motion and/or other metrics of breathing were detected during the waiting time period 1506 (or a similar time period without a waiting period following detection of an un-acceptable metric value), then the controller may return to monitoring and evaluating the acceptability of the trigger metric at block 1504.

If the controller determines at block 1508 that no motion or breathing metrics were detected during the waiting time period 1506, then the controller may proceed to block 1512 to evaluate whether the trigger metric is improving. In some implementations, the controller may determine whether the trigger metric is improving by obtaining a new trigger metric value (e.g., by obtaining a new measurement or by retrieving from a memory device one or more measurement values obtained after the value evaluated in block 1504) and evaluating whether the new trigger metric value (or values) has increased relative to the previous trigger metric value obtained at block 1504 prior to the waiting period 1506. For example, if the new trigger metric obtained at block 1512 is one percentage point or more improved relative to the previous measurement, then the metric may be deemed to be improving. In various implementations, the amount of improvement required for a “yes” answer at block 1512 may be a function of triggering indication. For instance, if there is a PETCO2 value recorded in the last 5 seconds or SpO2 has increased by at least 1%. As described above, some trigger metrics improve by increasing in value (e.g., SpO2, O₂A, DSR, RR) while others improve by decreasing (e.g., AUC_{Desat}.)

Therefore, if the controller determines that the metric is improving acceptably, then the controller may be instructed to return to monitoring and evaluating the acceptability of the trigger metric to detect declines in breathing quality. In some implementations, the determination of metric improvement at block 1512 may be based on multiple data samples obtained within a trailing period of time, such as all or several measurements within the previous 1 second to one minute or more.

In various implementations, an acceptable improvement magnitude at block 1512 may be determined based on the trigger metric being used. In the case of SpO2, an improvement of at least 1% may be adequate to establish an improving condition sufficient to return a “yes” result. In other implementations, a greater SpO2 improvement of 2% or more may be required in order to return a “yes” result at block 1512.

On the other hand, if the controller determines at block 1512 that the trigger metric is not improving sufficiently or that it is getting worse, the controller may be instructed to deliver a stimulus pulse of a first level at block 1516. In some implementations, a first level of stimulus pulse may be a first duration and a first electrical current. In some implementations, the first duration may be a duration of between about a quarter second, a half-second, one second, two seconds, three seconds, or more. A first level current may be about two milli-Amps to about five milli-Amps.

After delivering a first level stimulus, the controller may proceed to block 1518 to evaluate whether the trigger metric is improving at an acceptable rate. If not, then the controller may deliver a stimulus of a second level at block 1520. In some implementations, a second level of stimulus pulse may be a second duration longer than the first duration of the first-level pulse and/or a second electrical current that may be greater than the first electrical current of the first-level pulse. In some implementations, the second duration may be a duration of between about two seconds to about ten seconds or more, and in one particular implementation, the second duration stimulus may be on the order of about three seconds. A second level current may be about two milli-Amps to about ten milli-Amps. In various implementations, a second-level stimulus may differ from a first level stimulus in only duration, only current, or both duration and current. A second level electrical stimulus is generally defined as a stimulus with a greater total quantity of delivered energy than a first level electrical stimulus.

If the controller determines at block 1518 that the trigger metric is improving, then the controller may return to monitoring the trigger metric, optionally after waiting a period of time. Such a wait-time delay may ensure that the system does not deliver more than a desired number of stimulation events within a particular period of time. In some implementations, the period of time during which the controller may be instructed to wait before returning to monitoring may be on the order of a few seconds to a few minutes or more. For example, in some implementations, the wait time may be between about one minute and about 10 minutes or more, or in one particular implementation about five minutes.

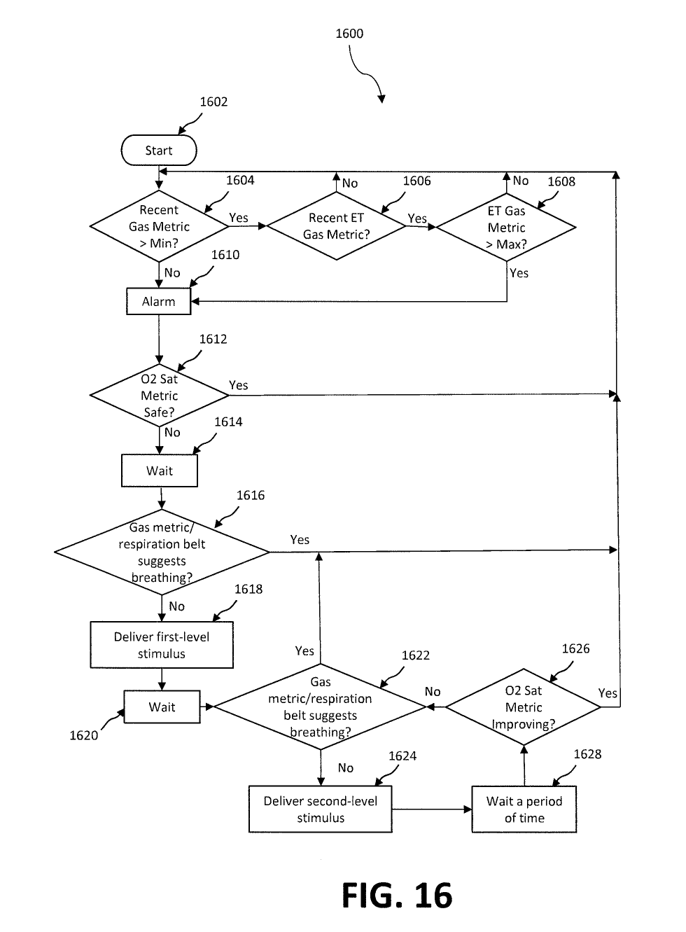

FIG. 16 illustrates a process 1600 for monitoring a patient and delivering an arousal stimulation based on both an oxygen saturation metric and a breathing metric. Portions of the process of FIG. 16 may be substantially similar to portions of the process 1500 of FIG. 15, including blocks 1614, 1616, 1618, 1624, and 1626.

The process 1600 may begin at block 1602 while the system monitors and stores data for the various oxygen saturation and breathing metrics. In various implementations, the breathing metric may be gases CO2, O2, or respiratory rate. Nonetheless, the process 1600 will be described with reference to a CO2 breathing gas measurement.

At block 1604, the system may evaluate a breathing rate and gas metric to determine whether the metric exceeds a minimum acceptable value threshold. For example, a minimum acceptable value of a CO2 measurement may be above 10 mmHg and the indication of a respiratory rate presence. If the gas metric is not above a minimum acceptable value, the process may proceed to block 1610 at which an audible and visible alarm may be initiated. In various implementations, an alarm condition may include an audible alarm, a visible alarm such as a displayed text warning a flashing light or other visible alarm, or others. In some implementations, an alarm condition may include transmitting a message to a remote device such as a nurse's station or a mobile device.

A CO2 or respiratory rate measurement below a minimum acceptable value may provide a more immediate indication of stopped breathing than SpO2 which tends to exhibit a substantial measurement latency (i.e., an SpO2 measurement may indicate physiological events that occurred sometime before the measurement was obtained and reported).

If the gas and breathing metrics exceed the minimum value, the process may proceed to block 1606 to determine whether an end-tidal measurement has been obtained within a recent period of time (e.g., within the last few seconds, from about one second to about 10 seconds, or in one particular implementation within about five seconds). If not, the process may return to monitoring metrics. If an end tidal measurement has been obtained within a recent time period, the process may proceed to block 1608 at which the end-tidal measurement and respiratory rate may be compared against a maximum acceptable threshold. For example, an end-tidal CO2 measurement of more than about 40 to 50 mmHg and respiratory rate more than 35 may indicate hypercarbia which may indicate disordered breathing.

If the gas metric does not exceed the maximum threshold, the process may return to monitoring metrics. If the gas metric does exceed the maximum threshold, the process may proceed to block 1610 at which an alarm condition may be initiated.

At block 1612, the process may evaluate a blood oxygen saturation metric such as SpO2, AUC_{Desat}, O₂A, or DSR (collectively referred to as “O2 Sat metrics” because all are obtained from oxygen saturation data.) Evaluation of the O2 Sat metric at block 1612 may include a determination of whether the metric indicates a safe or acceptable condition. For example, an SpO2 value or an O₂A value higher than an upper acceptable threshold (e.g., about 95%), a positive DSR value, or a zero AUC_{Desat} value may indicate a safe condition. If the SpO2 metric at block 1612 indicates a safe condition, the process may return to monitoring metrics.

If the O2 Sat metric at block 1612 does not indicate a safe condition, the process may wait for a period of time at block 1614 before proceeding to evaluating whether the gas metric indicates the presence of breathing and normal respiratory range at block 1616.

At block 1616, a gas metric and breath rate (e.g., CO2 or O2) may be evaluated to determine whether an end-tidal measurement of a measured gas and respiration rate is above a minimum acceptable threshold indicating that the patient is breathing. If this is true, then the system may return to monitoring metrics.

If the gas metric and/or breath rate is not above the minimum threshold, then the system may deliver a first-level stimulus at block 1618. After delivering a first-level stimulus, the controller may wait a period of time to allow for any physiologic delay in measurement or response to the delivered stimulus. Following any waiting time, the controller may proceed to block 1622 at which a gas metric may be evaluated to determine whether the patient is breathing as above.

If the gas metric at block 1622 does not indicate breathing, then a second-level stimulus may be delivered at block 1624. After delivering a second-level stimulus, the controller may proceed to block 1626 at which the controller may evaluate whether the O2 saturation metric is improving at an acceptable rate. If the O2 saturation metric is improving acceptably, then the controller may return to monitoring. If the O2 saturation metric is not improving acceptably, then the controller may return to block 1622 to evaluate whether a gas metric suggests breathing.

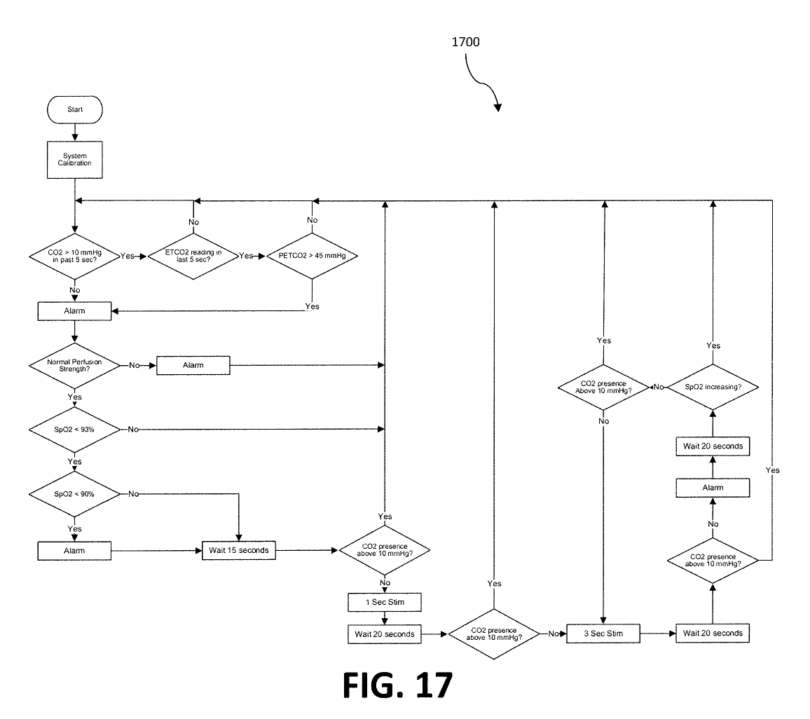

In some example implementations, a process for determining when to deliver stimulation may evaluate a measurement of exhaled carbon dioxide, a measurement of blood oxygen saturation (SpO2), and one or more measurements of motion such as the respiratory rate. FIG. 17 illustrates one particular example implementation process 1700.

In the particular implementation illustrated in FIG. 17, when the system is initiated, it may begin an automatic system calibration process to ambient air. The system may then check for a presence of CO2 in the gas sampling line by checking for a reading above 10 mmHg and breath rate above minimum. If the CO2 reading is below 10 mmHg for the last 5 seconds, the system may sound an alarm and continue through the loop. If the CO2 reading is above 10 mmHg and there has been an end tidal CO2 (PETCO2) value captured within the last 5 seconds above a designated threshold value (40 to 45 mmHg), the device may display an alarm message and continue through the loop.

If the system determines that the breath rate and PETCO2 value are normal (below the upper threshold value) it may return to the beginning of the loop. The system may then check the patient's perfusion as indicated by the pulse oximeter to determine whether the perfusion strength is within a normal range. Based on clinical implementation experience, a signal to noise ratio above 30% can be chosen. If the perfusion strength is not within a normal range, a warning message may be displayed, and the software may return to the beginning. If the perfusion strength is normal, then the controller may proceed to checking blood oxygen saturation levels. If the SpO2 is below the designated upper threshold value (e.g., 90%-93%), the system may check if the SpO2 is below a lower threshold (e.g., 89% or 90%). If the SpO2 is also below the lower threshold, the system may display an alarm message before continuing through the loop. If SpO2 is above the upper threshold value, the system may return to the beginning.

Once it is determined that SpO2 is below the lower threshold, the controller may administer a 15 second waiting period before continuing through the loop. The system may then check to determine whether a gas measurement indicates breathing (e.g., if a CO2 value of at least 10 mmHg is detected). If the gas metric indicates a quantity of gas indicated at a normal breathing rate, then the system may return to the beginning.

If the gas metric or breath rate does not indicate a quantity of sufficient normal range of values, a one-second stimulus may be administered to the patient via the stimulator electrodes. Following delivery of a one-second stimulus, the controller may wait for a period of time (e.g., about 20 second) before evaluating a gas metric to determine whether the patient is breathing (e.g., whether CO2 is present in an amount greater than 10 mmHg and breath rate is outside of normal range). If the gas metric indicates breathing, the system may return to the beginning of the loop.

If the gas metric does not indicate breathing within normal breath range, the controller may deliver a three-second stimulus, after which it may wait for a period of time (e.g., 20 seconds). Following a waiting time, the system may again evaluate a quantity of a gas or breath rate to determine whether the patient is breathing within in normal limits. If the gas measurement indicates breathing (within normal breath rate range), the system may return to the beginning of the loop, but if not, then an alarm may be triggered, the system may wait another waiting period, and the system may evaluate whether an oxygen saturation metric is improving (e.g., whether SpO2 is increasing by an acceptable amount).

If the oxygen saturation metric is improving, the system may return to the beginning of the loop. Otherwise, the system may again evaluate whether a breathing gas measurement indicates breathing. If so, the system may return to the top of the loop. If not, then the system may deliver another three-second stimulus.

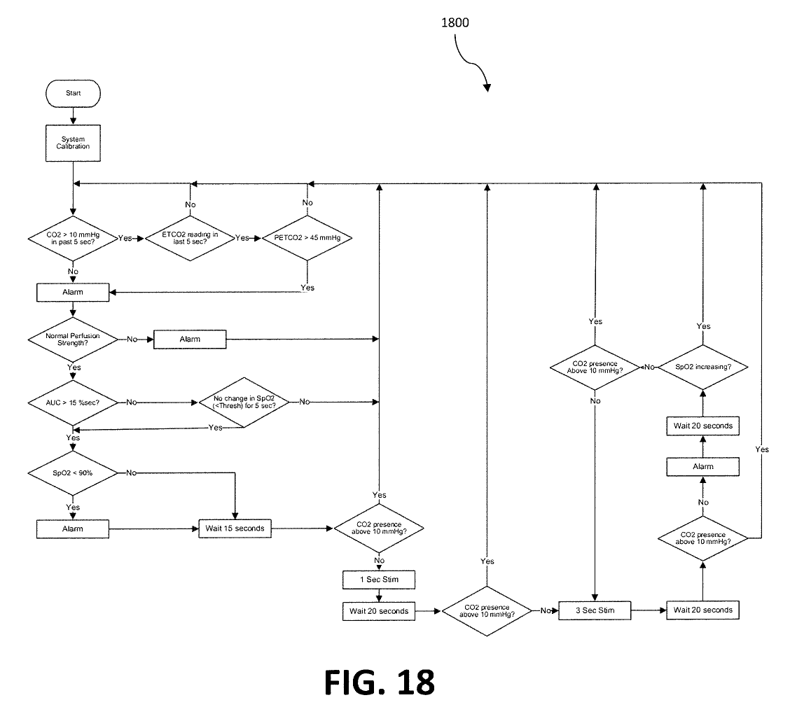

In another example implementation, a process for determining when to deliver stimulation may evaluate a measurement of exhaled carbon dioxide, an area-under-the-curve (AUC) metric based on a measurement of blood oxygen saturation (SpO2), and one or more measurements of motion such as breath rate. FIG. 18 illustrates one such example implementation process 1800.

In the particular implementation illustrated in FIG. 18, when the system is initiated, it may begin an automatic system calibration process. The system may then check for a presence of CO2 in the gas sampling line by checking for a reading above 10 mmHg in the past 5 seconds and breath rate above minimum. If the CO2 reading is below 10 mmHg for the last 5 seconds, the system may sound an alarm and continue through the loop. If the CO2 reading is above 10 mmHg and there has been an end tidal CO2 (PETCO2) value captured within the last 5 seconds above a designated upper threshold value (e.g., 40 to 45 mmHg), the device may display an alarm message and continue through the loop.

If the system determines that the breath rate and PETCO2 value are normal (below the upper threshold value) it may return to the beginning. The system may then check the patient's perfusion as indicated by the pulse oximeter to determine whether the perfusion strength is within a normal range. Based on clinical implementation experience, a signal to noise ratio above 30% can be chosen. If the perfusion strength is not within a normal range, a warning message may be displayed, and the software may return to the beginning. If the perfusion strength is normal, then the controller may proceed to checking the AUC value.

If the AUC is above the designated threshold value (e.g., 15 to 25% sec), the controller may continue through the loop. If AUC is below the threshold value, it may check to see if SpO2 has remained the same for at least 5 seconds. If SpO2 is unchanged, it may continue through the loop, else it may return to the beginning. If SpO2 is also below 90%, it may display an alarm message before continuing through the loop.

The system may then wait for a period of time (e.g., about 15 seconds). The system may then check to determine whether a gas measurement indicates breathing (e.g., if a CO2 value of at least 10 mmHg is detected). If the gas metric indicates a quantity of gas indicative of normal rate of breathing, then the system may return to the beginning.

If the gas or breath rate metric does not indicate a quantity of gas sufficient to suggest breathing, a 1 second stimulus may be administered to the patient via the stimulator electrodes. Following delivery of a one-second stimulus, the controller may wait for a period of time (e.g., about 20 second) before evaluating a gas metric to determine whether the patient is breathing (e.g., whether CO2 is present in an amount greater than 10 mmHg and breath rate is outside of normal range). If the gas metric indicates breathing, the system may return to the beginning of the loop.

If the gas metric does not indicate breathing, the controller may deliver a three-second stimulus, after which it may wait for a period of time (e.g., 20 seconds). Following a waiting time, the system may again evaluate a quantity of a gas and or breath rate to determine whether the patient is breathing. If the gas measurement indicates breathing, the system may return to the beginning of the loop, but if not, then an alarm may be triggered, the system may wait another waiting period, and the system may evaluate whether an oxygen saturation metric is improving (e.g., whether SpO2 is increasing by an acceptable amount).

If the oxygen saturation metric is improving, the system may return to the beginning of the loop. Otherwise, the system may again evaluate whether a breathing gas measurement indicates breathing. If so, the system may return to the top of the loop. If not, then the system may deliver another three-second stimulus.

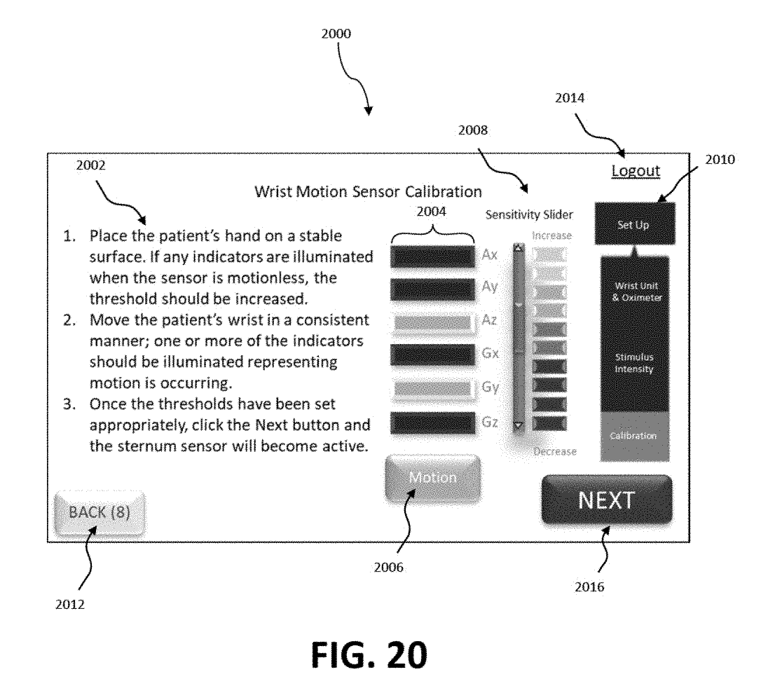

In some implementations, a monitor will be used with the breathing monitoring and arousal system. In some implementations, that monitor will be programmed with a graphical user interface that may be navigated through by a user. FIG. 20 shows a screen 2000 that may be used during the motion sensor calibration process. It may be used to set individual motion thresholds for patients 2010. Instructions 2002 may be placed on the screen for the calibration process. It is important that the sensors are properly calibrated for each patient as each person has a slightly different movement profile while breathing. The screen may display indicators 2004 that light up if the sensors sense motion 2006. The sensitivity slider 2008 may be used by the clinician to adjust sensor sensitivity appropriately for each patient. Motion may be detected if at least two of the sensor indicators 2004 light up. The screen may also display a back button 2012, a logout button 2014, and a next button 2016 for program navigation.

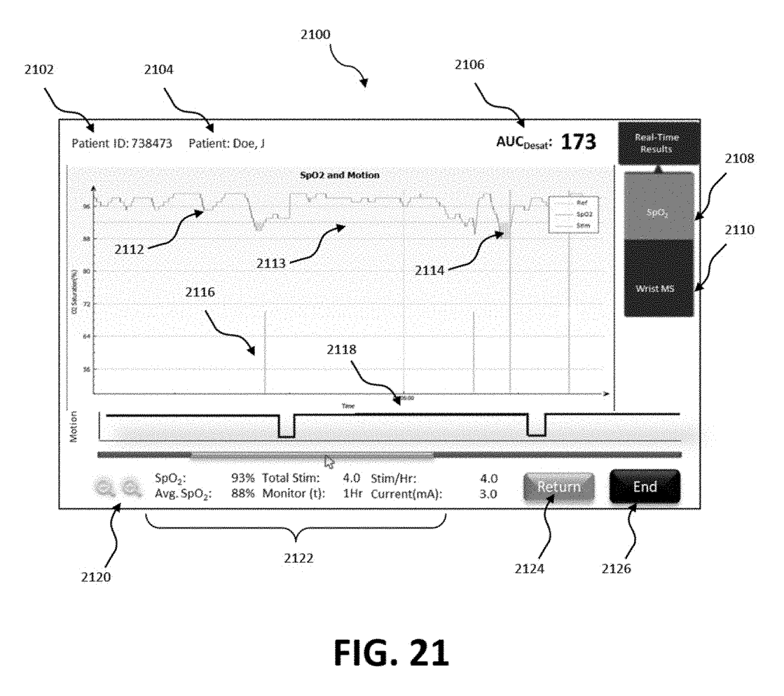

In some implementations, a monitor will display live data throughout the measuring session. FIG. 21 shows a potential screen 2100 for this purpose. The screen may display information about the patient, including the patient ID 2102 and the patient name 2104. The screen may record and display SpO2 waveforms 2112, real-time SpO2 measurements 2122, stimulation events 2116, reference SpO2 2113, and AUC 2106. Additionally, a binary waveform 2118 capturing the presence of motion may be displayed throughout the session. Graphically, AUC may be displayed as shaded regions 2114 on a graphs display. As more shaded area appears on the waveform display, the numerical AUC may increase on the screen and may keep a rolling record, or it may reset to zero whenever the SpO2 rises again above the threshold, depending on the implementation. The screen may display instantaneous and averaged measurements 2112 throughout the session. Instantaneous and averaged metrics may act as trigger metrics as discussed before. The user may be able to scroll through the waveforms on the screen and alter the zoom on the graphs 2120. The user may be able to switch between displays for SpO2 2108 and wrist motion 2110. The user may also navigate between different screens in the program by using the return 2124 and end 2126 buttons.

An exemplary system according to one implementation of the present disclosure includes a controller, a breathing sensor securable to a patient and configured to determine breathing data of the patient, a pulse oximetry sensor securable to the patient and configured to sense pulse oximetry data of the patient, and a stimulator securable to the patient and configured to stimulate to the patient. The controller is configured to execute instructions to obtain the breathing data of the patient from the breathing sensor, obtain the pulse oximetry data of the patient from the pulse oximetry sensor, determine, based on the breathing data of the patient and the pulse oximetry data of the patient, whether the patient is to be stimulated, and deliver, using the stimulator, stimulations to the patient, in response to determining that the patient is to be stimulated.

An exemplary system according to another implementation of the present disclosure includes a base unit including a user interface, a display, a carbon dioxide sensor, an electrical stimulation controller, a pulse oximetry controller, and a system controller. The system further includes a breath capture device securable to a patient's mouth or nose and connected to the base unit by a sample collection tube to provide breathing gas to the carbon dioxide sensor. The system also includes a wrist unit including stimulation electrodes extending from the housing, and a strap configured to secure the housing to the patient's wrist. The system further includes a finger clip configured to be secured to the patient's finger and including a pulse oximetry sensor in communication with the pulse oximetry controller. The system controller is configured to execute instructions to obtain the breathing gas data received from the carbon dioxide sensor, obtain pulse oximetry data from the pulse oximetry controller, determine, based on the breathing gas data and the pulse oximetry data, whether an electrical stimulus is to be delivered to the patient, and deliver the electrical stimulus to the patient using the stimulation electrodes, in response to determining that the electrical stimulus is to be delivered to the patient.

An exemplary system according to another implementation of the present disclosure includes a base unit including a user interface, a display, a respiration monitor belt, an electrical stimulation controller, a pulse oximetry controller, and a system controller. The system further includes a respiration rate capture device securable to a patient's chest area and connected to the base unit, and a wrist unit including stimulation electrodes extending from the housing, and a strap configured to secure the housing to the patient's wrist. The system also includes a finger clip configured to be secured to the patient's finger and including a pulse oximetry sensor in communication with the pulse oximetry controller. The system controller is configured to execute instructions to obtain the respiratory rate data received from the respiration monitor belt, obtain pulse oximetry data from the pulse oximetry controller; determine, based on the respiratory rate and the pulse oximetry data, whether an electrical stimulus is to be delivered to the patient, and deliver the electrical stimulus to the patient using the stimulation electrodes, in response to determining that the electrical stimulus is to be delivered to the patient.

An exemplary system according to another implementation of the present disclosure includes a system controller, a carbon dioxide sensor, an electrical stimulator, and a pulse oximetry measurement device. The system controller is configured to obtain breathing gas data of a patient using the carbon dioxide sensor, obtain pulse oximetry data of the patient using the pulse oximetry measurement device, determine, based on the breathing gas data and the pulse oximetry data, whether an electrical stimulus is to be delivered to the patient, and deliver an electrical stimulus to the patient using the electrical stimulator, in response to determining that the electrical stimulus is to be delivered to the patient.

An exemplary method according to one implementation of the present disclosure is used by a system including a controller, a breathing sensor securable to a patient and configured to determine breathing data of the patient, a pulse oximetry sensor securable to the patient and configured to sense pulse oximetry data of the patient, and a stimulator securable to the patient and configured to stimulate to the patient. The method includes obtaining the breathing data of the patient from the breathing sensor, obtaining the pulse oximetry data of the patient from the pulse oximetry sensor, determining, based on the breathing data of the patient and the pulse oximetry data of the patient, whether the patient is to be stimulated, and delivering, using the stimulator, stimulations to the patient, in response to determining that the patient is to be stimulated.

An exemplary method according to another implementation of the present disclosure is used by a system according to another implementation of the present disclosure includes a base unit including a user interface, a display, a carbon dioxide sensor, an electrical stimulation controller, a pulse oximetry controller, and a system controller. The system further includes a breath capture device securable to a patient's mouth or nose and connected to the base unit by a sample collection tube to provide breathing gas to the carbon dioxide sensor. The system also includes a wrist unit including a housing and stimulation electrodes extending from the housing, and a strap configured to secure the housing to the patient's wrist. The system further includes a finger clip configured to be secured to the patient's finger and including a pulse oximetry sensor in communication with the pulse oximetry controller. The method includes obtaining the breathing gas data received from the carbon dioxide sensor, obtaining pulse oximetry data from the pulse oximetry controller, determining, based on the breathing gas data and the pulse oximetry data, whether an electrical stimulus is to be delivered to the patient, and delivering the electrical stimulus to the patient using the stimulation electrodes, in response to determining that the electrical stimulus is to be delivered to the patient.

An exemplary method according to another implementation of the present disclosure includes measuring components of gases exhaled by a patient, measuring a blood oxygen saturation level of the patient, measuring the patient's motion, determining that a quantity of at least one of the components of the gases exhaled by the patient is outside of a range for a first period of time, detecting that the blood oxygen saturation level of the patient is below a saturation threshold for a second period of time, and delivering an electrical stimulus to the patient, in response to the detecting and the determining.

From the above description, it is manifest that various techniques can be used for implementing the concepts described in the present application without departing from the scope of those concepts. Moreover, while the concepts have been described with specific reference to certain implementations, a person of ordinary skill in the art would recognize that changes can be made in form and detail without departing from the scope of those concepts. As such, the described implementations are to be considered in all respects as illustrative and not restrictive. It should also be understood that the present application is not limited to the particular implementations described above, but many rearrangements, modifications, and substitutions are possible without departing from the scope of the present disclosure.

What is claimed is:

1. A system comprising:
a controller;
a breathing sensor securable to a patient and configured to determine breathing data of the patient;
a pulse oximetry sensor securable to the patient and configured to sense pulse oximetry data of the patient;
a stimulator securable to the patient and configured to stimulate the patient;
the controller configured to execute instructions to:
obtain the breathing data of the patient from the breathing sensor;
obtain the pulse oximetry data of the patient from the pulse oximetry sensor;
determine, based on the breathing data of the patient, and the pulse oximetry data of the patient, whether the patient is to be stimulated; and
deliver, using the stimulator, stimulations to the patient, in response to determining that the patient is to be stimulated.

2. The system of claim 1, wherein the breathing sensor includes a respiration monitor belt securable to a chest area or abdomen of the patient.

3. The system of claim 1, wherein the breathing sensor includes a face mask securable to a mouth and/or a nose of the patient, and further includes a breath sampling tube connected to a gas monitoring unit.

4. The system of claim 3, wherein the breathing sensor includes nose cannula, and further includes a breath sampling tube connected to a gas monitoring unit.

5. The system of claim 3, wherein the breathing data is analyzed to determine an average of measured end-tidal carbon dioxide values obtained during a time period falls outside of an acceptable range.

6. The system of claim 3, wherein the breathing data comprises an end-tidal oxygen value below a threshold value.

7. The system of claim 3, wherein the breathing data comprises an end-tidal carbon dioxide value for comparison with a threshold.

8. The system of claim 3, wherein the breathing data comprises an area-under-the-curve analysis summation of oxygen saturations below a threshold.

9. The system of claim 1 further comprising a motion sensor securable to the patient and configured to sense motion data of the patient, wherein the controller is further configured to execute instructions to obtain the motion data of the patient from the motion sensor, and wherein determining whether the patient is to be stimulated is further based on the motion data of the patient.

10. The system of claim 1 further comprising another motion sensor securable to a sternum of the patient, wherein the controller is further configured to increase a duration of the stimulations when the motion data from the motion sensor or another motion sensor indicates motion within a pre-determined monitoring time period.

11. The system of claim 10, wherein the controller is further configured to determine that a first motion is sensed by the motion sensor when a first signal received from the motion sensor exceeds a first motion signal threshold, and that a second motion is sensed by the second motion sensor when a second signal received from the another motion sensor exceeds a second motion signal threshold, the second motion signal threshold being greater than the first motion signal threshold.

12. The system of claim 1, wherein the stimulator includes stimulation electrodes extending from the unit for attachment to the patient.

13. The system of claim 1, wherein the pulse oximetry sensor is a finger clip configured to be secured to a finger of the patient.

14. The system of claim 1, wherein the pulse oximetry sensor is an ear clip configured to be secured to an ear lobe of the patient.

15. The system of claim 1, wherein the stimulator is configured to deliver electrical pulses using a variable voltage and/or a constant current.

16. The system of claim 1, wherein the breathing data and the pulse oximetry data are obtained over a period of time.

17. The system of claim 1, wherein the system controller is configured to execute instructions to generate an audible alarm based on an analysis of the breathing data and the pulse oximetry data using one or more thresholds.

18. The system of claim 1, wherein the pulse oximetry data comprises a result of an area-under-the-curve analysis summation of differences between a threshold oxygen saturation and measured oxygen saturation below the threshold multiplied by the duration of oxygen saturation below the threshold.

19. The system of claim 1, wherein the pulse oximetry data comprises an oxygen saturation area based on an average of oxygen saturation values over a period of one minute.

20. The system of claim 1, wherein the pulse oximetry data comprises a count of events during which oxygen saturation falls below a threshold value within a time period.

21. The system of claim 1, wherein the pulse oximetry data comprises a desaturation rate calculated as a rate of change of blood oxygen saturation below a threshold oxygen saturation value.

22. The system of claim 1, wherein the controller is further configured to not deliver stimulations while a skin impedance measured across stimulation electrodes falls outside of a predetermined range.

23. The system of claim 1, wherein the controller is further configured to deliver the stimulations based on a combination of two or more metrics derived from the breathing data and the pulse oximetry data.

24. The system of claim 1, wherein system is further configured to receive a user input, via a user interface of the system, selecting a stimulation waveform.
